(19) United States
(12) Patent Application Publication
(10) Pub. No.: US 2022/0296427 A1
(43) Pub. Date: Sep. 22, 2022

(54) A SECUREMENT DEVICE

(71) Applicant: 3M INNOVATIVE PROPERTIES COMPANY, St. Paul, MN (US)

(72) Inventor: Ganesh Prasad Durgam Kannan, Bangalore (IN)

(21) Appl. No.: 17/641,737

(22) PCT Filed: Oct. 2, 2020
(86) PCT No.: PCT/IB2020/059247

§ 371 Date: Mar. 9, 2022

(30) Foreign Application Priority Data
Oct. 3, 2019 (IN) 201941039958

(51) Int. Cl. A61F 13/02

(52) U.S. Cl. CPC A61F 13/0236; A61F 2013/00412

(57) ABSTRACT

The securement device eliminates the drawbacks of prior venous cannulas securement method by providing various plates, substrates and adhesive layers in the securement device which secures the article firmly onto the underlying substrate arresting movement in all axis that does not need any additional form of securement.

### FIELD

The disclosure relates to a securement device for mounting an article to a conformable surface, such as a medical device on skin and to a method for securing an article to a conformable surface, such as a medical device on the skin surface of the patient.

### BACKGROUND

Various medical devices are attached to a patient. For example, tubing, monitors, sensors, or catheters are secured to skin. To limit irritation, dislodgement, and potential exposure to infection the medical devices should be securely attached to the patient. Adhesives and adhesive tapes are commonly used to secure devices to skin. Very strong adhesives can cause trauma to skin upon removal. A very gentle adhesive will remove from the skin easily, but might not have sufficient strength to secure the medical device.

One common medical device that is secured to a patient's skin is an intravenous venous catheter. Few types of intravenous catheter include central line catheter and peripheral line catheter. A peripheral venous catheter often referred to as an intravenous cannula is a flexible hollow plastic tube that is inserted in a peripheral vein most commonly the metacarpal vein of the hand or cephalic or basilic vein of the upper limb forearm. The peripheral venous catheterization involves piercing of a vein or artery of a patient with needle carrying cannula called as catheter and the cannula is slowly slid over the needle and into the punctured blood vessel. Subsequently, the needle should be removed from the cannula and the cannula is closed with a cap. The cannula would be then secured by means of an additional form of securement by tapping or with adhesive patches and is connected to a connector for supplying an intravenous fluid to the patient. Most frequent complications in catheterization procedures are due to repeated movements of the cannula inside the vein resulting in friction of the cannula against the entry site, dislodgement of the cannula and accidental removal. This may result in repeated microtrauma and laceration of skin edges leading to phlebitis, infection and pain for the patients.

Improper securement of peripheral venous catheter to the skin allows the catheter to move within the vein, which increases the incidence of peripheral venous catheter dislodgement, mechanical phlebitis, infiltration, leakage and infection. This movement results in peripheral venous catheter failure, an interruption to intravenous therapy and the need to re-site the peripheral venous catheter. Repeated re-siting of peripheral venous catheter can lead to venous access difficulties, including the need for more frequent peripheral venous catheter re-sites or for a central venous catheter, and causing interruption to the delivery of IV therapy and a potential increase in the duration of hospital stay and healthcare costs.

U.S. Pat. No. 8,591,471: A catheter assembly having a catheter with a longitudinal body having a transverse attachable detachable fastening member thereunder comprising a pliable polymeric element having a top side and a bottom side with the top side having a longitudinal groove fixedly attached to the catheter's longitudinal body with the bottom side having an adhesive layer covered by a removable cover so that when removed the catheter assembly can be adhesively attached to a recipient's skin and when desirous of removal can be peelably removed from said recipient's skin.

U.S. Pat. No. 8,157,770: A stabilizing device (and method of use) for insertion and maintenance of a catheter or cannula maintains a sterile insertion site and is simple and easy to construct and use, yet optimally maintains the catheter or cannula in place. A first stabilizing element includes a pair of bilateral wings and a catheter or cannula receiving element generally between the wings. A proximal stabilizing element—desirably two distinct and spaced pieces of tape—is operatively connected to the wings and has a sterile adhesive for affixing the wings to a patient to provide stability during needle removal and substantially for the duration that the catheter or cannula is inserted into a patient. A lateral stabilizing element—desirably two pieces of tape—is distinct and spaced from the proximal stabilizing element, to provide primarily lateral and medial stability to a catheter or cannula in a patient. A release sheet may cover the tape adhesive, or the tape may have a pattern of adhesive and non-adhesive sections cooperating with each other. Butterfly catheter, or angiocatheter, embodiments are particularly desirable.

EP2814552: The application relates to a two-part overlapping dressing for securing a cannula during intravenous catheterization on the skin surface of a patient. The dressing comprises a first flexible adhesive sheet (10) provided with an opening (16) spaced from all its peripheral edges; and a second flexible adhesive sheet (12). The opening (16) in the first sheet comprises a slit (18), one end of which terminates in an enlarged aperture (20). The second sheet (12) is dimensioned such that, when it is aligned with and adhered against the first sheet (10) in use, it is capable of overlapping the full length of the slit (18) so as to reduce the opening (16) to the size of the enlarged aperture (20). The apparatus allows for a more secure fitting of the cannula whilst minimising the risk of infection.

U.S. Pat. No. 5,087,248: An adhesive patch for anchoring an intravenous needle to the skin of a patient at the transcutaneous insertion site is disclosed in which a patch of continuous flexible asymmetrically elastic sheet material having one side covered with a layer of biocompatible adhesive is provided substantially shaped as large and small abutting parallel rectangles integrally joined parallel to the easy axis of elasticity and adapted to engage an intravenous needle. The small rectangle is configured such that a portion can disengage from the larger rectangle and fold on itself as a tab and adhesively engage the periphery of the hub of the needle and wherein the larger rectangle has a central shaped cut out facing the small rectangle and is adapted to fold over the tab and adhesively attach to the skin thereby attaching the entire assembly to the skin. A layer of peel able release paper adhering to said adhesive is provided for packaging.

EP1307253: An anchoring system for securing a dialysis catheter to a patient comprises an anchor pad and a retainer. The anchor pad is attached to the skin of the patient by an adhesive layer on one side of the pad. The retainer is disposed upon the surface of the anchor pad opposite the adhesive layer and includes a base and a cover and a adhesive spot. A groove upon the base is arranged to receive a Y-site portion of a dialysis catheter where the lumens merge distal to the insertion site. A post also protrudes from the base to the cover at a position which will be disposed between the two distal branches of the dialysis catheter. The cover closes over the base, securing the Y-site between the groove and the post. Closing the cover also places at least a portion of the catheter in contact with the adhesive spot. This contact between the retainer, adhesive spot and catheter thereby inhibits inadvertent motion of the catheter upon the patient. A latch mechanism is disposed upon the retainer to maintain the cover in a closed position over the Y-site of the catheter on the base.

U.S. Pat. No. 3,910,272: A cannula for the introduction of a flexible catheter comprises a cannula having a wing of a foil material joined to each side and it includes a cannula protective cap at a spaced location from a guard cap for the catheter. An intermediate bacteria-proof shell encloses the cannula between the protective cap and the guard cap, and it has two separable portions which are arranged in juxtaposition over the wings. The protective shell includes projecting portions to permit it to be spread open to cause a corresponding movement of the wings and the breaking open of the cannula. The protective shell is made preferably of a single moulded part which is joined together at a hinge, but which includes a separate foil hinge and which include inwardly projecting dogging elements which engage against the wings after the lower portion of the housing is engaged around the cannula. The half shell portions are each provided with inter-engageable projections and recesses so that the shell portions are locked together to form a bacteria-proof joint.

U.S. Pat. No. 3,910,272 describes the design with bacteria-poof closure ensuring perfectly sterile manipulation.

According to the conventional methodology, the vein would be initially identified and the patient's skin should be cleaned with alcohol wipe and allowed to dry. Subsequently, the cannula is taken from its sterile packaging and then the needle cover is removed without touching the needle. As a next step the cannula with needle, the bevel looking upwards at 30 degree, is advanced into the peripheral vein until a flashback of blood is seen in the hub at the back of the cannula. Finally, the clinician opens a sterile pack containing the securement patch which may have a hypoallergenic plastic tape and such like parts which would be applied over the cannula in a crisscross configuration for securement. According to this existing technology, dressing requires the engagement of both hands of the clinician to apply and sometimes more than one clinician to apply depending upon the patient. Moreover, the rate of dislodging of the cannula secured by this existing methodology is a higher.

### SUMMARY

The disclosed securement device has an extensible substrate with an adhesive surface and two opposing tabs on the extensible substrate that are stretched in opposing directions to easily remove the adhesive surface from the underlying substrate. This securement device is well suited for use on underlying substrates that are conformable, such as skin. Conformable substrates may be any suitable non-rigid surface that will slide, compress, or stretch, and stretching the extensible substrate in opposing provides stability when releasing the adhesive surface from the underlying conformable substrate. This stretching to release the adhesive surface from the underlying substrate allows for a strong adhesive to be used on the securement device.

In one embodiment, the securement device secures an existing medical device, such as a venous catheter, firmly onto the skin to limit movement in all axis.

In one embodiment, the securement device further includes cross tapes that enable the clinician to secure existing peripheral venous catheter firmly to the skin to limit movement in all axis.

In one embodiment, the securement device is secured to the peripheral venous catheter which enables the clinician to secure the assembly firmly to the skin to limit movement in all axis.

### BRIEF DESCRIPTION OF THE DRAWINGS

The following drawings illustrate the various embodiments and parts of the claimed devices.

FIG. 1 illustrates an article in orthogonal view.

FIG. 2 illustrates a securement device in orthogonal view.

FIG. 3 illustrates the various parts of the securement device in exploded view.

FIG. 4 illustrates the resistance to movement of the securement device connected with the article in all axis.

FIG. 5 illustrates rear view of the article mounted on the securement device.

FIG. 6A illustrates the adhesive adherence to the underlying substrate.

FIG. 6B illustrates the tabs being pulled in opposing direction for stretch release.

### DETAILED DESCRIPTION

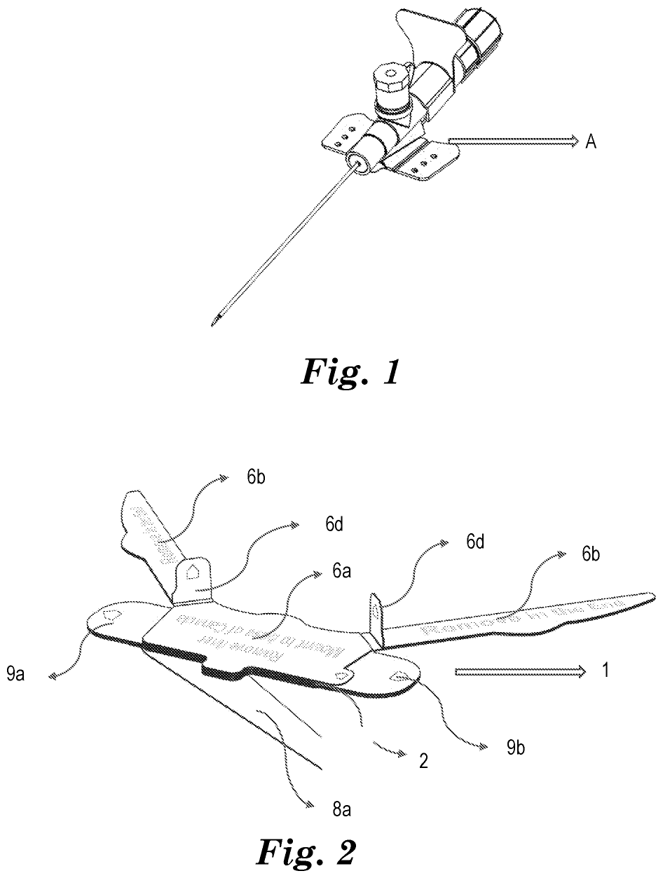

The one of the objectives of the device (1) is to secure an article (A) onto the surface of the underlying substrates. The underlying substrate may be conformable surface that is any suitable non-rigid surface that will slide, compress, or stretch, such as skin, foam, hydrogel, etc or can flex and not interfere with the stretch removable characteristics of the device (1), although it can be stretchable or not. However, underlying substrate may be in any form. For example, the underlying substrate may be a surface of an article that may not be linear e.g., a curved surface. The article may be, but it is not limited to medical devices such as IV catheter (such as article (A) shown in FIG. 1). In other embodiments, the article (A) may be a hook for holding articles, or any other object that needs to be adhered onto the underlying substrate. The securement device (1) for securing the article (A) as shown in FIG. 2, includes an extensible substrate (2) with a first surface and a second surface. A first adhesive (3) is provided on at least a portion of the first surface and a second adhesive (7) is provided on at least a portion of the second surface. The securement device (1) includes a first tab (9a) at a first end of the extensible substrate (2) and a second tab (9b) at a second end of the extensible substrate (2), opposite to the first end.

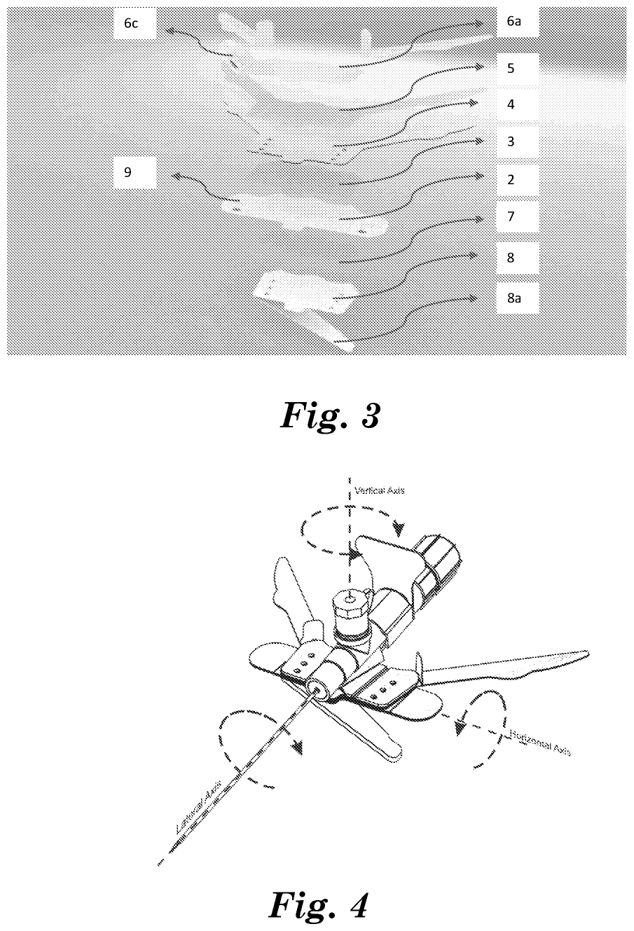

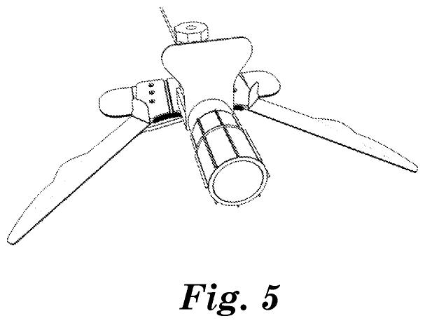

In one embodiment, the first tab (9a) and second tab (9b) are free of exposed adhesive. The first tab (9a) and the second tab (9b) are used for removing the securement device (1) from the underlying substrate in detail later with respect to FIGS. 3-6A and 6B.

Considering the example of the article (A) being an intravenous catheter, as per standard clinical protocols, the article (A) is inserted through the underlying substrate such as skin, and once it is in a vein: the liner/needle is removed and the article is pressed onto the skin. The article (A) is placed exactly at the position where it entered, such as the vein. The adhesive between the extensible substrate (2) and the underlying substrate hold the article (A) in place.

The complete underside portion of the article (A) may be in contact with the underlying substrate (2). Subsequently in an embodiment, the two cross tapes having cover/liner (6a, 6b) on the long tail of the device (1) can be placed over the needle entry point in a crossed manner and pressed them onto the underlying substrate so as to secure the article (A) on the skin. This completes the securement of the article (A). No additional patches or tapes are required to hold the article (A). In one embodiment, the above configuration allows to collect flash back blood, change to IV lines or infuse drugs into the article (A) without the fear of dislodging it. Further, even though the article (A) in this example is an intravenous catheter, the device (1) can be used to position other medical devices (e.g. wearable medical devices) or articles or electronic devices on a conformable surface (such as, skin or clothing) in various other embodiments. Thus, the device (1)(broadly explained here) can be customized for any number of applications including medical, industrial, and consumer products.

The device (1) has multiple parts as shown in FIG. 3. The extensible substrate (2) may be made of a stretchable material which assists in the easy applying and removing of the device (1) that can conform to undulated and conformable surfaces.

In one aspect, the top side of the extensible substrate (2) has first adhesive (3) with superior adhesive properties. A substrate with plurality of optional cross tapes (4) or at least a pair of cross tapes is bonded on the adhesive layer (3) at the top side of the extensible substrate (2). In one embodiment, a third adhesive layer (5) secures any type of material used to make article (A), and this layer (5) is available at the top side of the substrate with cross tapes (4). An adhesive layer/fourth adhesive layer (5a) is on the top portion of the cross tapes (4). The adhesive layers (5, 5a) at the top side of the substrate with cross tapes (4) are protected with plurality of protective cover/liner (6a, 6b) at the top.

For example, the first protective cover (6a) at the top side of the substrate with cross tapes (4), where the article (A) shall be fixed, have a tab (6c) on one end so that the tab (6c) can be pulled out easily before installing the article (A) in case of retrofit application. After securement of the article (A) to the adhesive layer (5) at the top side of the substrate with cross tapes (4), the bonding ensures no movement of the article (A). Any slight difference in size between the extensible substrate (2), the substrate with the cross plates (4) and the bottom side of the article (A) will not have any impact on the movement as the adhesives used are strong enough to adhere article (A) larger in size than the extensible substrate (2). Similarly, the adhesives used are not limited to the weight of the article (A) as the adhesives used can hold all types of existing articles, such as medical devices or other articles based on different applications or need. The adhesive on these tapes have antimicrobial properties that protect and secure the cannulated site in case of intravenous application.

In another embodiment, if the device (1) is retrofit the protective cover (6a) is peeled to expose the adhesive layer (5) at the top side of the substrate with cross tapes (4). Then, the bottom side of the article (A) is placed onto the adhesive layer (5) at the top side of the substrate with cross tapes (4) and it is pressed gently. Now, the securement device (1) is a part of the article (A).

The bottom side of the extensible substrate (2) has a second adhesive layer (7) which is a stretch release adhesive layer. The adhesive layer (7) used in the bottom side of the extensible substrate (2) may be a skin friendly adhesive. The said adhesive layer (7) has stretching property i.e., it retains its tack and adhesion property as long as its initial shape is intact. The two tabs (9a, 9b) of the extensible substrate (2) provided at opposite ends helps in stretching the adhesive layer (7) thereby releasing the securement device (1) and article (A) from the underlying substrate. Explaining in another way, pulling on the extensible substrate (2) using the tabs (9a and 9b) causes the extensible substrate (2) to elongate or stretch. Stretching reduces the volume of the extensible substrate (2) in the region touching the underlying substrate such as, the conformable surface and facilitates release of the extensible substrate (2) from the underlying substrate. Pulling the tabs (9a and 9b) can release the adhesive layers from the substrate (2) if the adhesive layer has sufficient cohesive strength. The adhesive layer has sufficient shear strength and adhesive holding power for the specific applications, its adhesive holding power to any substrate preferably being less than its cohesive strength, such that the adhesive layer will not be separate from the backing when the backing is subjected to stretching. The adhesive layer is also preferably highly extensible. In an embodiment, the extensible substrate (2) typically can be stretched in a first direction (often the first direction is length-wise) without breaking or snapping under the stretch releasing conditions. After the extensible substrate (2) is stretched along a major axis and is released from the substrate, substantially no adhesive residue may remain on the underlying substrate.

A protective cover (8) protects the adhesive layer (7) at the bottom side of the extensible substrate (2) while not in use. The protective cover (8) at the bottom side has a tab (8a) that extends like a long arm. The tab (8a) capable of being folded back to one side of the cover at the bottom. After, the article (A) fitted with the securement device (1) is placed over the underlying substrate, the tab (8a) is pulled to remove the protective cover (8). Since the tab (8a) is designed in such a way that it folds back during the pull operation it delaminates from the adhesive layer (7) in face down position making it simpler for one-handed operation. Finally, a simple manual press on the article (A) or the securement device (1) will firmly secure the article (A) and the securement device (1) to the underlying substrate.

Subsequent to the completion of securing the article (A) and the securement device (1) to the underlying substrate, such as skin, the tab (6d) present in second protective cover (6b) of each cross tape is pulled to expose the fourth adhesive layer (5a). The cross tapes are then crossed over the article (A) entry point by the adhesive layer on the top of the plurality of cross tapes/fourth adhesive layer. On applying simple manual pressure across the cross tapes, the cross tapes will seal the article (A) entry point and provide additional restriction to the movement of the securement device (1) and the article (A) in all directions/axis such as, vertical axis, horizontal axis and lateral axis as shown in FIG. 4. The crossing over of cross tapes over the cannula entry point also substantially reduces the rate of microbial infection.

The fourth adhesive layer used at the cross tapes does not leave any residue on the cannula entry point when the securement device (1) along with the article (A) is removed. The shape of the adhesive layer can be deformed by extending/stretching/expanding.

In one embodiment, colour changing cross tapes can be used as a different alternative to the normal cross tapes to detect microbial activity. For example, if microbes start to grow in the cannulation site, the colour of tapes may change indicating microbe growth thereby providing warning to the patient or user.

In another aspect, the article (A) is directly bonded over the adhesive available on the top side of the extensible substrate (2) of the device (1). Moreover, this aspect may not comprise the substrate with cross tapes, the adhesive layer over the substrate with the cross tapes and other parts related to the cross tapes.

In yet another aspect, an embodiment herein provides a method of securing a device to a substrate with the securement device (1). The method comprises the steps of: i) applying the article (A) to the first adhesive (3); and ii) applying the second adhesive (7) to the underlying substrate (as shown in FIG. 3). In one embodiment, the method further comprises the steps of: removing a first protective cover (6a) enveloping a third adhesive layer (5), wherein the third adhesive layer (5) is secured on at least a portion of a substrate (4) positioned on the first adhesive (3); securing the article (A) to the third adhesive layer (5); removing a third protective cover (8) enveloping the second adhesive (7); and securing the article (A) fitted to the extensible substrate (2) to the underlying substrate by tapping the article (A) or the extensible substrate (2) thereby exerting pressure to the second adhesive (7). In another embodiment, the method further comprises the steps of: removing second protective cover (6b) enveloping a fourth adhesive layer (5a) secured on at least a portion of the substrate (4) having plurality of cross tapes and crossing the plurality of cross tapes across the article (A) for securing the article with securement device (1) to the underlying substrate. Even though, the method of securing the device to the substrate is described in a particular order herein according to one embodiment, the steps of the method can be performed in any other different order and are not be construed as limiting this disclosure in any manner.

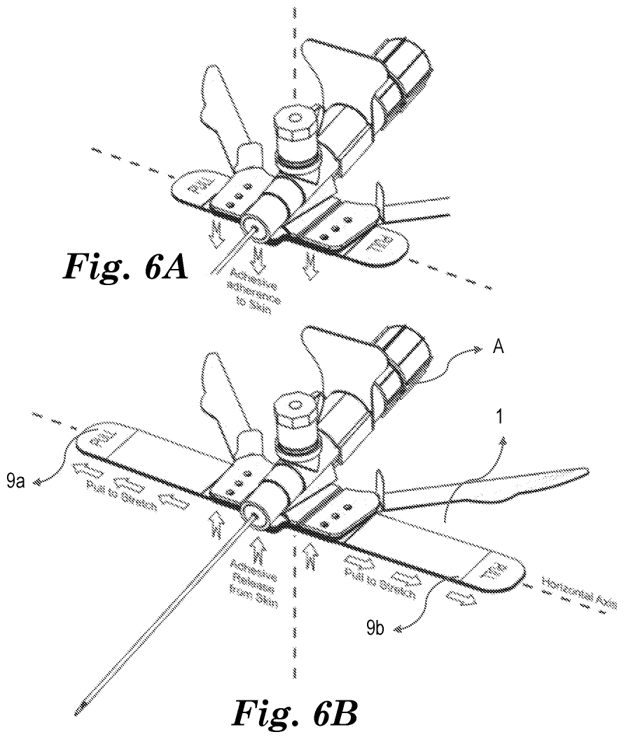

Moving on to FIGS. 6A and 6B, an embodiment herein provides a method of removing the adhesive securement device (1) from the underlying substrate. The method includes the steps of: (i) stretching the first tab (9a) in a first direction; and (ii) stretching the second tab (9b) in a second direction, which is opposite from the first direction. Once stretched, the extensible substrate (2) gets released from the underlying substrate. In an embodiment, the first tab (9a) and the second tab (9b) may be pulled along the plane of the bond to stretch release the device (1). The direction of the stretching the first tab (9a) and the second (9b) are shown by arrows in FIG. 6B and the releasing of the extensible substrate (2) from the underlying substrate is shown using the arrows in perpendicular direction in FIG. 6B. In an embodiment, the extensible substrate (2) may be stretched lengthwise in a direction substantially parallel to the plane of the substrate surface. The direction of stretching of the extensible substrate (2) may be at any preferred angle to the surface of the substrate. For example, the direction of stretching of the extensible substrate (2) may be less than 35° to the surface of the substrate. However, such tabs are preferably pulled at about 45° up to right angles (i.e., normal) to the substrate and surface to which the adhesive article is attached, although this angle may be reduced to near zero (i.e., substantially in the plane of the adhesive bond) if the surface is not rigid (e.g., skin).

The tabs 9(a) and 9(b) are shown in FIGS. 6A and 6B are shown to have a particular shape and size, however, it may be envisioned that the tabs 9(a) and 9(b) can have different shapes and configuration.

In an embodiment, the extensible substrate (2) can be provided in any useful form including, e.g., tape, strip, sheet (e.g., perforated sheet), label, roll, web, disc, and kit (e.g., an object for mounting and the extensible substrate used to mount the object). Likewise, multiple extensible substrate can be provided in any suitable form including, e.g., tape, strip, sheet (e.g., perforated sheet), label, roll, web, disc, kit, stack, tablet, and combinations thereof in any suitable package including, for example, dispenser, bag, box, and carton.

### LIST OF REFERENCE NUMERALS

A—Article
1—Securement device
2— Base plate/extensible substrate
3— Adhesive layer at top side of the base plate/first adhesive layer
4— Substrate with plurality of cross tapes.
5— Adhesive layer on the top of the substrate with plurality of cross tapes/third adhesive layer
5a—Adhesive layer on the top of the plurality of cross tapes/fourth adhesive layer
6a— Protective cover covering a part of the adhesive layer at the top of the substrate with plurality of cross tapes/first protective cover
6b—Protective cover covering the adhesive layer at the top of the cross tapes/second protective cover
6c— Tab present in protective cover of (6a).
6d— Tab present in the protective cover (6b).
7— Adhesive layer at the bottom side of the base plate/second adhesive layer
8—Protective cover covering the adhesive layer at the bottom side of the base plate/third protective cover
8a—Tab present in the protective cover (8).
9a, 9b—Tabs at the opposite ends of the extensible substrate for stretching the adhesive layer at the bottom side of the extensible substrate.

1. An adhesive securement device for securing an article comprises:
an extensible substrate with a first surface and a second surface;
a first adhesive on at least a portion of the first surface;
a second adhesive on at least a portion of the second surface;
a first tab at a first end of the extensible substrate; and
a second tab at a second end of the extensible substrate, opposite the first end.

2. The adhesive securement device as claimed in claim 1, wherein the first tab and second tab are free of exposed adhesive.

3. The adhesive securement device as claimed in claim 1, further comprising a first and second cross tapes extending from the extensible substrate and secured to the first adhesive.

4. The adhesive securement device as claimed in claim 1, further comprising a third adhesive layer on the first cross tape and a fourth adhesive layer on the second cross tape.

5. The adhesive securement device as claimed in claim 4, further comprising a first protective cover over the third adhesive layer and a second protective cover over the fourth adhesive layer.

6. The adhesive securement device as claimed in claim 5, wherein the first protective cover and the second protective cover comprises peeling tabs respectively to assist in removing the first protective cover and the second protective cover.

7. The adhesive securement device as claimed in claim 1, wherein the first adhesive, the second adhesive are stretch releasable.

8. The adhesive securement device as claimed in claim 1, further comprising a third protective cover comprising a peeling tab to assist removing the third protective cover for exposing the second adhesive and fixing the extensible substrate onto an underlying substrate.

9. The adhesive securement device as claimed in claim 1, wherein the first tab and second tab are pulled for releasing the extensible substrate from the underlying substrate.

10. The adhesive securement device as claimed in as claimed in claim 1, wherein the article is a medical device, and the underlying substrate is skin.

11. A method of securing the article to an underlying substrate with the securement device as claimed in claim 1 comprising the steps of:
applying the article to the first adhesive; and
applying the second adhesive to an underlying substrate.

12. The method as claimed in claim 11, comprising the steps of:
removing a first protective cover enveloping a third adhesive layer, wherein the third adhesive layer is secured on at least a portion of a substrate positioned on the first adhesive;
securing the article to the third adhesive layer;
removing a third protective cover enveloping the second adhesive; and
securing the article fitted to the extensible substrate to the underlying substrate by tapping the article or the extensible substrate thereby exerting pressure to the second adhesive.

13. The method as claimed in claim 11, wherein securing the adhesive securement device further comprising the steps of removing second protective cover enveloping a fourth adhesive layer secured on at least a portion of the substrate having plurality of cross tapes and crossing the plurality of cross tapes across the article.

14. A method of removing the adhesive securement device, as claimed in claim 1, from the underlying substrate, the method comprising:
stretching the first tab in a first direction; and
stretching the second tab in a second direction, which is opposite from the first direction.
